(19) United States
(12) Patent Application Publication
(10) Pub. No.: US 2009/0118801 A1
(43) Pub. Date: May 7, 2009

(54) Discharge lamp having spectral power distribution shift and methods of making the same

(76) Inventors: John Tulk, Prospect, CT (US); Donald L. Smith, Tucson, AZ (US); Kathleen J. Goldschmidt, Huntington, CT (US); Patricia E. Reykdal, Tucson, AZ (US)

Correspondence Address: EDWARDS ANGELL PALMER & DODGE LLP, P.O. BOX 55874, BOSTON, MA, 02205, US

(21) Appl. No.: 11/663,315

(22) PCT Filed: Sep. 20, 2005
(86) PCT No.: PCT/US2005/033781

§ 371 Date: Aug. 22, 2008

Related U.S. Application Data
(60) Provisional application No. 60/611,379, filed on Sep. 20, 2004.

(51) Int. Cl. A61N 5/06; H01J 63/04; C09K 11/54

(52) U.S. Cl. 607/94; 313/485; 252/301.6 P

(57) ABSTRACT

Disclosed are discharge lamps for use in tanning applications and methods of making the same. The lamps include, among other elements, an elongated vitreous tube which has an outer periphery and axially opposed first and second ends that define an axial length for the tube. A coating is applied on the interior of the tube for emitting ultraviolet radiation in tanning wavelengths when a voltage to the lamp. The coating is specifically formulated so that the ultraviolet radiation emitted from the lamp in a range of about 320 nm to about 340 nm is less than or equal to about 10 percent of the ultraviolet radiation emitted from the lamp in a range of about 280 nm to about 400 nm.

### CROSS REFERENCE TO RELATED APPLICATIONS

This application claims priority to U.S. Provisional Patent Application Ser. No. 60/611,379, filed Sep. 20, 2004, entitled “DISCHARGE LAMP HAVING SPECTRAL POWER DISTRIBUTION SHIFT,” the disclosure of which is herein incorporated by reference in its entirety.

### BACKGROUND OF THE INVENTION

1. Field of the Invention

The present disclosure relates generally to discharge lamps having phosphor coatings that emit skin tanning radiation, and more particularly, to discharge lamps that provide ultraviolet radiation within certain regions of the ultraviolet spectrum which have been found to improve skin tanning while also reducing the potential harmful effects associated with prolonged exposure to ultraviolet radiation.

2. Background of the Related Art

Discharge lamps of the fluorescent type that produce artificial skin tanning, defined as the darkening of one's skin through exposure to ultraviolet radiation, have been known for some time. Tanning through use of such discharge lamps has been steadily increasing in popularity in the United States since the late 1970s. Tanning lamps and tanning equipment used in the United States must comply with very specific regulations enforced by an agency of the Food and Drug Administration which restrict certain ultraviolet lamp characteristics mainly to protect consumers from possible harm due to prolonged exposure to ultraviolet radiation.

Most currently used tanning lamps produce a spectrum of ultraviolet light which is similar to that of the sun. The sun emits three kinds of ultraviolet (UV) rays, UVA, UVB and UVC. UVC, at 100 nanometers (nm) to 280 nm, is the shortest and widely considered to be the most harmful wavelength of UV rays, but it is virtually blocked by the Earth's ozone layer and pollution. UVB is the medium wavelength, from 280-320 nm, and although overexposure to UVB has been found to cause erythema (sunburn), a controlled amount is necessary to initiate tanning in the skin. UVA is the longest wavelength, from 320-400 nm, and has been found to be responsible for the completion of the tanning process (i.e., via oxidation or darkening of the pigment (melanin), as discussed in further detail herein). The sun is not selective in the proportions of UVA and UVB emitted. Therefore, by exposure to the sun, the skin is made vulnerable to amounts of UVB that can cause erythema, as well as other types of damage to the skin. In this regard, tanning with discharge lamps is advantageous to the sun since the current lamps can be made to only provide the small amount of UVB necessary to initiate the tanning process, while at the same time, providing the UVA needed to complete the tanning process.

However, each person's skin reacts differently to ultraviolet radiation exposure, with the reaction being dependent upon genetically determined factors, such as the amount of melanin pigment already in the skin naturally and the capability of the person's skin to produce additional melanin, which is referred to as facultative pigmentation.

Melanin itself is the dark pigment found in the retina, hair and skin, except for the palms of the hands, soles of the feet and lips. Without the protection afforded by the melanin pigment, a person's skin would burn when exposed to ultraviolet radiation. As stated above, the skin includes naturally occurring melanin pigment and produces additional melanin via special cells called melanocytes, which are located deep within the outer layer of the skin. When the melanocytes are stimulated by ultraviolet light, they utilize an amino acid called tyrosine to produce the pigment melanin. Since the melanocytes are only able to absorb ultraviolet light of approximately 260-320 nanometers, UVB radiation is needed to achieve melanin production. UVA radiation which has a wavelength of approximately 320-400 nanometers can formulate melanin, but only when there is enough photosensitizing material already in the skin to trigger a UVB reaction. With the presence of UVB, melanocytes are stimulated to divide, creating more pigment cells. During this time, the epidermis thickens to form additional protection, a condition referred to as acanthosis.

In the beginning stages of melanin production, the skin has very little melanin or radiation protection capabilities. As a result, UVA radiation is not blocked by melanin pigments and penetrates deep within the skin causing damage to the corium. Damage to this layer of the epidermis hastens aging and destruction of collagen and connective tissue. A UVA burn can be much more damaging because it is not felt due to its deep penetration.

In order for the pigmentation process to be effective, melanin granules must be oxidized or darkened, which requires a dose of UVA. Consequently, exposure to UVB radiation functions to create melanin pigment, while UVA exposure ensures the oxidation of the pigment. Together, the proper combination of UV exposure operates to create a light-protection mechanism.

Even though discharge lamps emit controlled amounts of UVA and UVB, there are still potentially harmful effects that can result from prolonged UV exposure. The damaging effects of UV on skin consist principally of cellular damage and alterations in immunologic function. Long-term effects include photo-aging, DNA damage and carcinogenesis.

Recent studies have shown that much of the harmful effects from prolonged UV exposure are caused by UVA radiation having a wavelength between 320 nm and 340 nm.

Thus, there is a need for a discharge lamp for use in tanning applications that has spectral characteristics, which provide the appropriate amounts of UVA and UVB to cause tanning, while also reducing some of the potentially harmful effects due to prolonged UV exposure by reducing the amount of UV rays having wavelengths between 320 nm and 340 mm.

### SUMMARY OF THE INVENTION

The subject invention is directed to new and useful discharge lamps that solve the problems described above. In particular, this invention relates generally to low pressure mercury vapor discharge lamps of the fluorescent type having a particular type phosphor coating(s) which is adapted to emit skin tanning radiation having a preferred spectra when excited by the UV radiation generated from the mercury vapor discharge. Lamps constructed in accordance with the present disclosure have spectral power distributions that provide satisfactory skin tanning with less of the aforementioned undesirable effects of UV exposure, such as for example, photo-aging.

Exemplary lamps constructed in accordance with the present invention provide a controlled amount of UVB radiation in the approximate 280 nm to 320 nm UV region of the spectrum and a controlled amount of UVA radiation in the approximate 340 nm to 400 nm UV region of the spectrum (hereinafter referred to as being “UVA1”), so that tanning occurs, while also reducing the amount of UV radiation in the approximate 320 nm to 340 nm UV region of the spectrum (hereinafter referred to as being “UVA2”) in comparison with prior tanning lamps.

A representative embodiment of the present disclosure is directed to a discharge lamp for use in tanning applications which includes, among other elements, an elongated vitreous tube that has an outer periphery and axially opposed first and second ends. A first electrode assembly is associated with the first end of the tube and a second electrode assembly is associated with the second end of the tube. Additionally, a coating is applied on the interior of the tube for emitting ultraviolet radiation in tanning wavelengths when a voltage is applied across the first and second electrodes. During operation, the ultraviolet radiation emitted from the lamp in a range of about 320 nm to about 340 nm is less than or equal to about 10 percent of the ultraviolet radiation emitted from the lamp in a range of about 280 nm to about 400 nm. Preferably, the ultraviolet radiation emitted from the lamp in the range of UVA2 range is less than about 1 percent of the total ultraviolet radiation emitted from the lamp in the UVA and UVB spectrum.

It is envisioned that in certain embodiments, the ultraviolet radiation emitted from the lamp in a range of about 340 nm to about 400 nm is greater than about 85 percent of the ultraviolet radiation emitted from the lamp in a range of about 280 nm to about 400 nm. Moreover, it is preferable that the ultraviolet radiation emitted from the lamp in a range of about 280 nm to about 320 nm is less than about 10 percent of the ultraviolet radiation emitted from the lamp. Still further, in certain lamps, the ultraviolet radiation emitted from the lamp in the UVB spectrum is preferably less than about 1.5 percent of the total ultraviolet radiation emitted from the lamp in both the UVA and UVB spectra.

In certain embodiments, the spectral power distribution is also adjusted so shift a larger percentage of the energy emitted in the UVB spectrum to the range of 280 nm to 302 nm (hereinafter referred to as the “UVB2”) from the range the 302 nm to 320 nm (hereinafter referred to as the “UVB1”). Such a energy shift also reduces the harmful effects from prolonged UV exposure, such as photo-aging, and provides more effective photon energy. In such embodiments, for example, the ultraviolet radiation emitted from the lamp in the UVB2 range is greater than about 5 percent of the ultraviolet radiation emitted from the lamp in the UVB spectrum. Preferably, the ultraviolet radiation emitted from the UVB2 range is greater than about 10 percent of the ultraviolet radiation emitted from the UVB range.

It is also envisioned that in certain embodiments of the present invention, the ultraviolet radiation emitted from the lamp in the UVA spectrum is greater than about 98 percent of the total ultraviolet radiation emitted from the lamp. Moreover, the ultraviolet radiation emitted from the lamp in the UVB spectrum is less than about 1.5 percent of the ultraviolet radiation emitted from the lamp.

The present disclosure is also directed to a device for effectuating tanning of a person's skin which includes a housing and at least one discharge lamp assembly. The housing has a body portion and a base portion, the body portion defining an internal tanning chamber which is adapted and configured for receiving a person.

The discharge lamp assembly is disposed within the internal tanning chamber and includes an elongated vitreous tube having an outer periphery and axially opposed first and second ends which define an axial length for the tube therebetween. A first electrode assembly is associated with the first end of the tube and a second electrode assembly is associated with the second end of the tube. Additionally, a coating is applied on the interior of the tube for emitting ultraviolet radiation in tanning wavelengths when a voltage is applied across the first and second electrodes. During operation, the ultraviolet radiation emitted from the lamp in a range of about 320 nm to about 340 nm is less than or equal to about 10 percent of the ultraviolet radiation emitted from the lamp in a range of about 280 nm to about 400 nm. Preferably, the ultraviolet radiation emitted from the lamp in the range of about 320 nm to about 340 nm is less than about 1 percent of the ultraviolet radiation emitted from the lamp in a range of about 280 nm to about 400 nm.

The present disclosure is also directed to a method of exposing a person to ultraviolet radiation comprising the steps of: a) positioning a discharge lamp assembly in proximity to a person, the discharge lamp being constructed in accordance with the teachings of this disclosure and b) energizing said discharge lamp assembly for a period of time to effect skin tanning of said person.

The present disclosure is also directed to a phosphor composition for use in a discharge lamp which includes europium activated strontium tetraborate; and thallium activated calcium zinc orthophosphate. In a preferred embodiment, the phosphor composition includes at least about 86% by weight of europium activated strontium tetraborate.

In alternative embodiments, the phosphor composition further includes lead activated barium silicate. It is envisioned that in certain constructions the composition includes about 59% by weight europium activated strontium tetraborate and about 13% by weight thallium activated calcium zinc orthophosphate. Alternatively, the composition includes about 62% by weight europium activated strontium tetraborate and about 4% by weight thallium activated calcium zinc orthophosphate. Still further, the composition can be formed to include about 65% by weight europium activated strontium tetraborate and about 6% by weight thallium activated calcium zinc orthophosphate. Although certain phosphor combinations are disclosed herein above, those skilled in the art will readily appreciated that alternative compositions of the listed phosphors can be used without departing from the inventive aspects of the present disclosure.

The elongated tubes used the present invention are filled with a rare gas, such as argon, and a drop of mercury. The vitreous tubes can have an outer periphery which is smooth or include a helical groove path formed therein over at least a portion of its axial length, similar to the lamps disclosed in U.S. Pat. Nos. 6,943,361 and 6,777,702, which are herein incorporated by reference in their entirety. Moreover, the phosphor coating can be applied to the lamp in a single coating with the desired chemical composition or a multi-layer coating, as know to those skilled in the art. Additionally, the coating(s) can be applied along the entire length of the lamp or over a portion of the length, as described in U.S. Pat. No. 6,919,676, which is herein incorporated by reference in its entirety.

The present disclosure is also directed to a method for fabricating a discharge lamp having a coated region. The representative method includes the steps of:
a) washing a vitreous tube with hot water;
b) drying the washed tube:
c) applying a phosphor coat which imparts UV spectral characteristics that provide UVA2 emissions of less than or equal to about 10% to a desired thickness over the full length of the tube;
d) baking the phosphor base coated tube in an oven;
e) sealing the tube with filament carrying mounts at both ends;
f) exhausting of the tube which includes evacuation of impurities;
g) filling with desired gas at a desired pressure;
h) dosing with mercury and sealing the discharge; and
i) vacuum sealing the mercury-containing tube.

A variation of the aforementioned method can be to seal the discharge prior to dosing with mercury. It is further envisioned that the method can include the steps of:
a) applying a reflector coating to the tube over a desired reflector angle, thickness and length;
b) baking the reflector coated tube to remove the binders used to faceplate the coating operation and leave only phosphor particles adhered to the bulb wall; wherein the reflector coat is applied and the coated tube is baked prior to applying the phosphor coat.

These and other aspects of the system and method of the subject invention will become more readily apparent to those having ordinary skill in the art from the following detailed description of the invention taken in conjunction with the figures and appended material.

### BRIEF DESCRIPTION OF THE DRAWINGS

FIG. 1 is a elevational view of a lamp constructed in accordance with an embodiment of the present invention;

FIG. 2a is a side elevational view showing an end of the lamp of FIG. 1;

FIG. 2b is a partial cross-sectional view of the lamp of FIG. 1 taken along cut line 2b-2b;

FIG. 3 provides a spectral irradiance curve and irradiance data for a prior art tanning lamp;

FIG. 4 provides a spectral irradiance curve and irradiance data for a lamp constructed in accordance with an embodiment of the present invention and using coating no. 1;

FIG. 5 provides a spectral irradiance curve and irradiance data for a lamp constructed in accordance with an embodiment of the present invention and using coating no. 2;

FIG. 6 provides a spectral irradiance curve and irradiance data for a lamp constructed in accordance with an embodiment of the present invention and using coating no. 3; and

FIG. 7 provides a spectral irradiance curve and irradiance data for a lamp constructed in accordance with an embodiment of the present invention and using coating no. 4.

### DETAIL DESCRIPTION OF PREFERRED EMBODIMENTS

Reference is now made to the accompanying figures for the purpose of describing, in detail, preferred and exemplary embodiments of the present disclosure. The figures and detailed description are provided to describe and illustrate examples in which the disclosed subject matter may be made and used, and are not intended to limit the scope thereof.

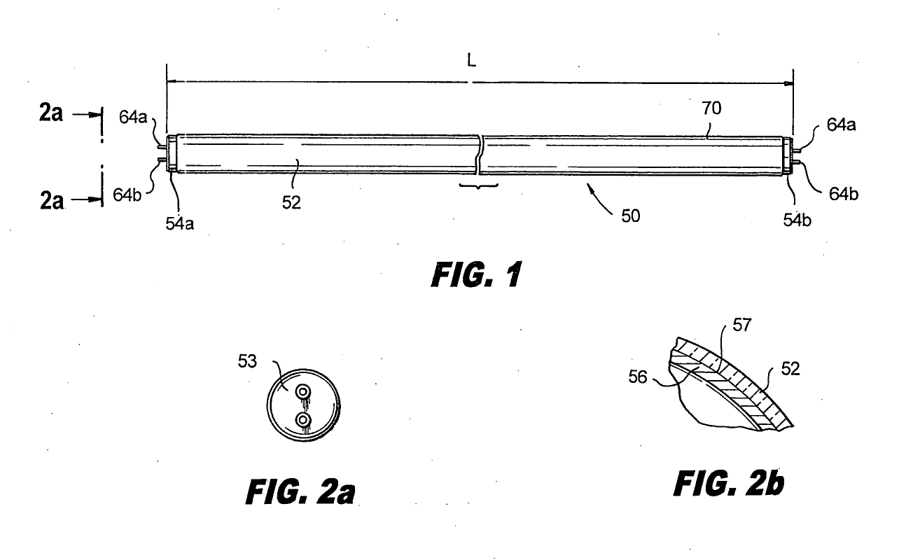

Referring now to the accompanying figure, there is illustrated in FIGS. 1, 2a and 2b a discharge lamp constructed in accordance with an exemplary embodiment of the present disclosure and designated generally by reference numeral 50. Discharge lamp 50 includes a vitreous tube 52, first and second end seals 64a and 64b, respectively, defining an enclosed region 53 extending longitudinally therein. Tube 52 has an outer periphery 70 which extends axially between the first and second end seals 64a and 64b. In a typical tanning application, the overall length of tube 52 is approximately 72 inches or 6 feet, but it should be readily apparent to those skilled in the art that the length of tube 12 can be modified to be other lengths as desired. It also should be appreciated that tube 52, need not be a straight tube, such as for example, certain face tanning lamps

A first electrode assembly is associated with the first end seal 54a and a second electrode assembly is associated with the second end seal 54b. Each electrode assembly typically includes pins 64a and 64b, which electrically communicate with corresponding electrical contacts associated with a lamp assembly. In an alternate embodiment, pins 64a and 64b can be replaced with a recessed double contact base or any other suitable electrical communication mechanism or arrangement, as will be readily appreciated by those skilled in the art.

Tube 52 has a phosphor coating 56 applied to interior surface 57, which may be disposed over a reflective coating as well. Tube 52 also has a drop of mercury disposed within central core enclosed region 53. In the embodiment shown herein, the phosphor coating 56 extends substantially over the entire length and inner circumference of surface 57 of tube 52. As will be readily apparent to those skilled in the art to which the present disclosure appertains, the length in which the coating is applied and the thickness and phosphor characteristics of coating 56 can be selectively adjusted based on the intended lighting application. Additionally, lamp 50 can include more than one region and coatings, or over-coatings and/or a different coating material or combination of materials. Moreover, the thickness of coating 56 and/or over-coating(s) may be varied to further achieve desired light application objectives.

In operation, an alternating current is applied to the pins 64a and 64b, which increases the temperature of the electrodes and causes the emission of electrons therefrom. These electrons are accelerated by the voltage across the tube 52 until they collide with the mercury atoms, causing them to be ionized and excited. When the mercury atoms return to their normal state, mercury spectral lines in both the visible and ultraviolet region are generated. The ultraviolet radiation excites phosphor coating 56 to luminance. The resulting output is not only much higher than that obtained from the mercury lines alone, but also results in a continuous spectrum with colors dependent upon the phosphors used.

The material or materials which are used in coating 56 and the thickness thereof are selected so as to adjust and/or control the spectral power distribution and level of the luminous intensity delivered by lamp 50, among other things. Additionally, coating 56 or another coating applied to interior surface 57 can include material that fluoresces in the visible light spectrum so as to provide light of coloration which is distinct to that region of the lamp. Since the UV radiation produced by the lamp is not visible, it may be desirable in a tanning application, for example, to provide a mechanism for indicating which region of the lamp emits light of a differing wavelength, e.g., based on the presence and/or characteristics of coating 56 and any over-coatings disposed thereon, and should be positioned over the face of a user.

In the present invention, the phosphors used for coating 56 are selected so that lamp 50 emits UV radiation that, in comparison with prior art lamps, is less in intensity in the UVA2 region. Preferably, coating 56 is configured so that lamp 10 also emits increased UV radiation intensity within the UVA1 region and shifts radiation in the UVB region to the lower UVB2 wavelength.

In addition, the present invention provides lamps with a tanning time (Tₜ), (i.e., the time it takes to deliver an optimal dose of tanning photons or “tanning power”), which is less than the time to reach the maximum irradiance that a lamp can deliver in its maximum timer interval (Tₑ), (i.e., technically, the time to reach 4.0 MED under standardized test conditions, wherein 4.0 MED is the maximum allowable dose). Tₑ is also known as the “sun burning power.”

Table 1 below illustrates the difference in spectral output by UV region between the current lamps, old lamps, the sun and lamps such as lamp 50 which as been constructed in accordance with the present disclosure.

TABLE 1
Percentage of Photons per UV region

| | | | | The Present |
|---|---|---|---|---|
| Parameter | Sun | Old Lamps | Current Lamps | Invention |
| UVC | 0 | 4 | 0 | 0 |
| UVB | 4.3 | 60 | 4.0 | <1.5 |
| UVA | 95.7 | 36 | 96.0 | >98.5 |
| UVA2 | 18.7 | 28 | 20.0 | <10.0 |
| UVA1 | 77.0 | 8 | 76.0 | >88.5 |

As the above table indicates, an exemplary lamp constructed in accordance with the present invention can be configured to emit less than 1.5% of its photons in the UVB range, less than 10% of its photons in the UVA2 region and greater than 88.5% of its photons in the UVA1 region. Thus, in comparison with the sun, current lamps and old lamps, a lamp constructed in accordance with the present invention increases the tanning photons in the UVA1 region while decreasing the photons in the UVA2 region, which is suspected of being the primary cause of UV exposure related photo-aging, among other things.

Table 2 below provides non-limiting exemplary coatings and phosphor compositions for use with lamps constructed in accordance with the present disclosure, wherein the phosphors are strontium tetraborate europium activated [SrB₄O₇:Eu], calcium zinc orthophosphate thallium activated [(CaZn)₃(PO₄)₂:Tl], and barium silicate lead activated [BaSi₂O₅:Pb]. Those skilled in the art will readily appreciated that other phosphors can be used in the composition which provide individually or in combination the desired output spectrum for the lamp.

TABLE 2
Exemplary Coatings and Phosphor Compositions
of the Present Invention

| Phosphor | Coating 1 | Coating 2 | Coating 3 | Coating 4 |
|---|---|---|---|---|
| SrB₄O₇: Eu | 86.6% | 58.9% | 62.2% | 65% |
| (CaZn)₃(PO₄)₂: Tl | 13.4% | 13.4% | 3.8% | 6% |
| BaSi₂O₅: Pb | None | 27.8% | 34% | 29% |

While exemplary coatings having spectral power distribution which, among other things, provide limited UVA2 radiation, have been described with respect to various specific embodiments, it should also be understood that the foregoing is only illustrative of exemplary and preferred embodiments, as well as principles of the subject invention, and that various modifications can be made by those skilled in the art without departing from the scope and spirit of the invention. In particular, it should be understood that other compositions and phosphors or phosphor blends can be used in the coatings to deliver the desired UV output in accordance with the present invention.

Therefore, the described embodiments should not be considered as limiting of the present invention in any way. Accordingly, the present disclosure embraces alternatives, modifications and variations of the present invention as fall and incorporated by reference into the subject application.

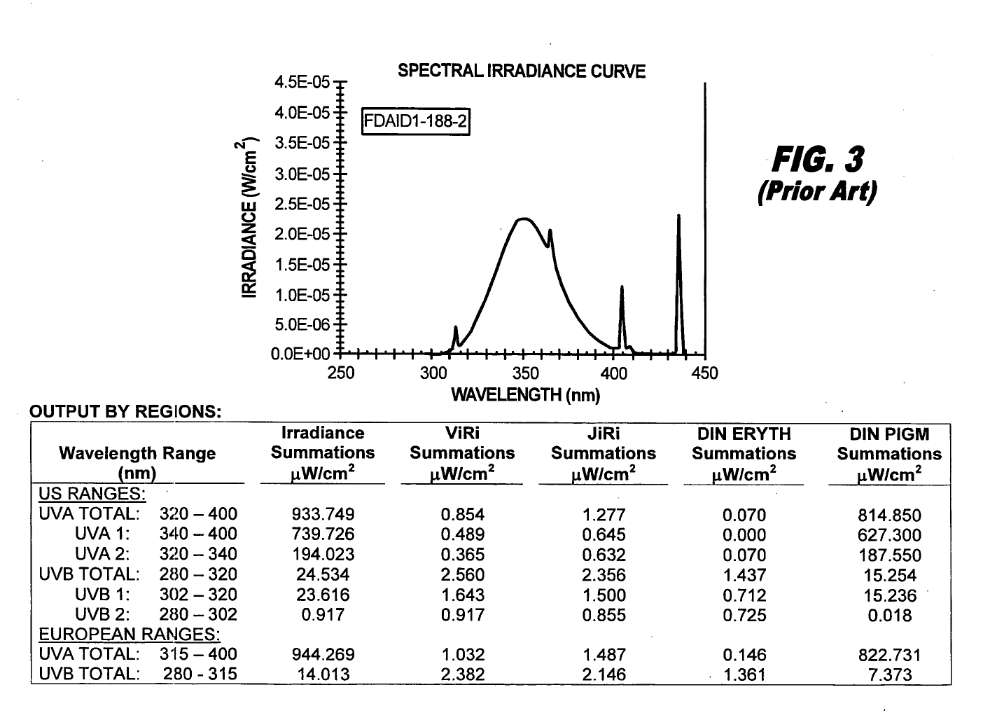

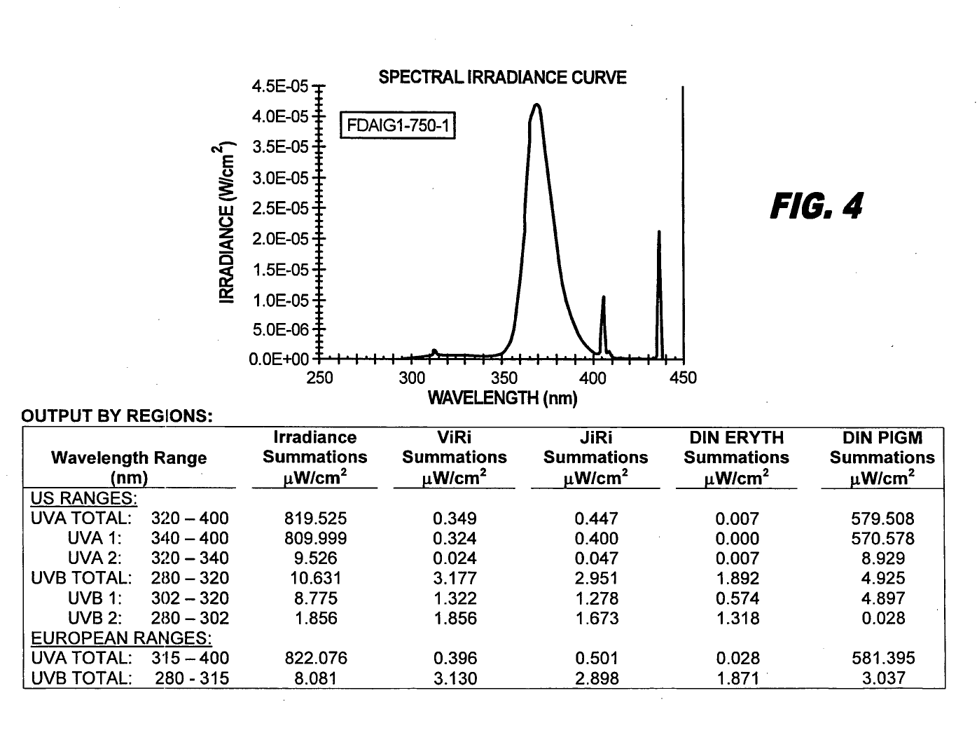

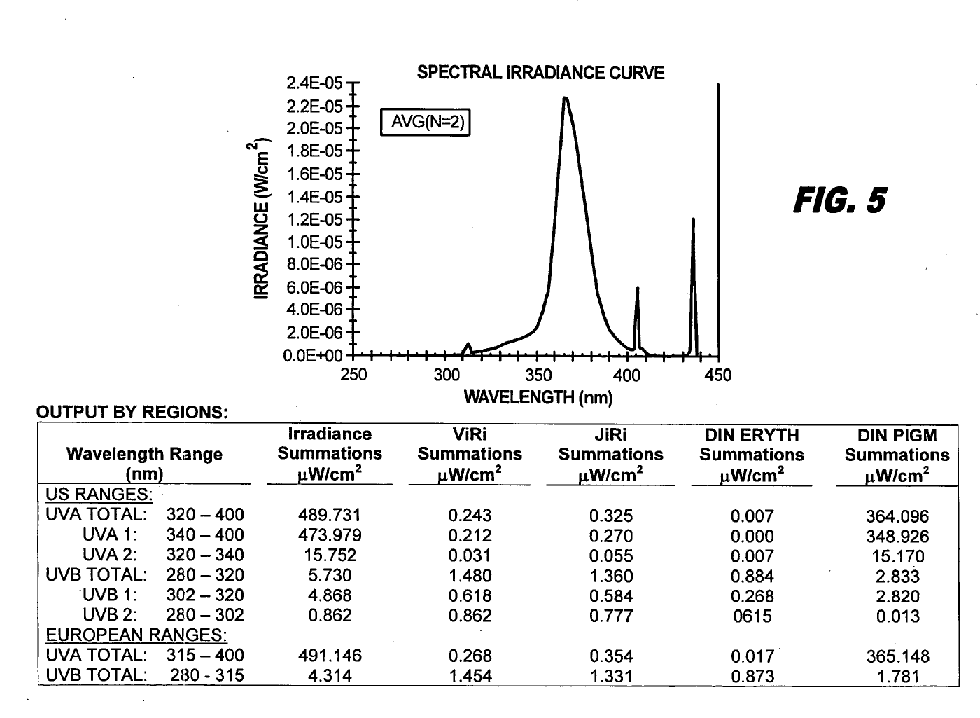

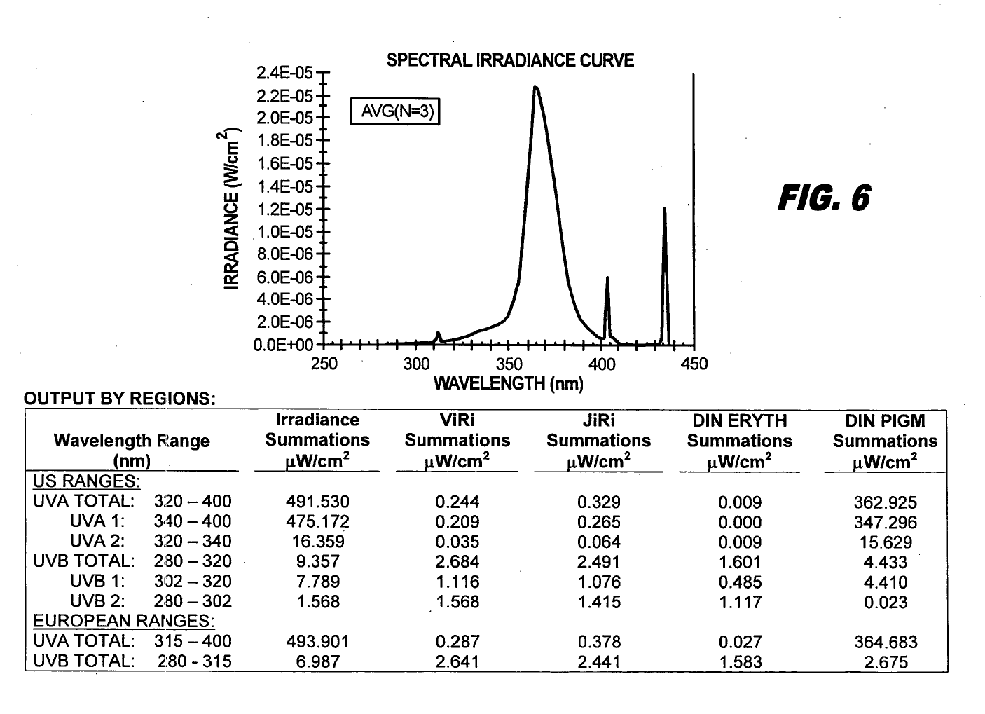

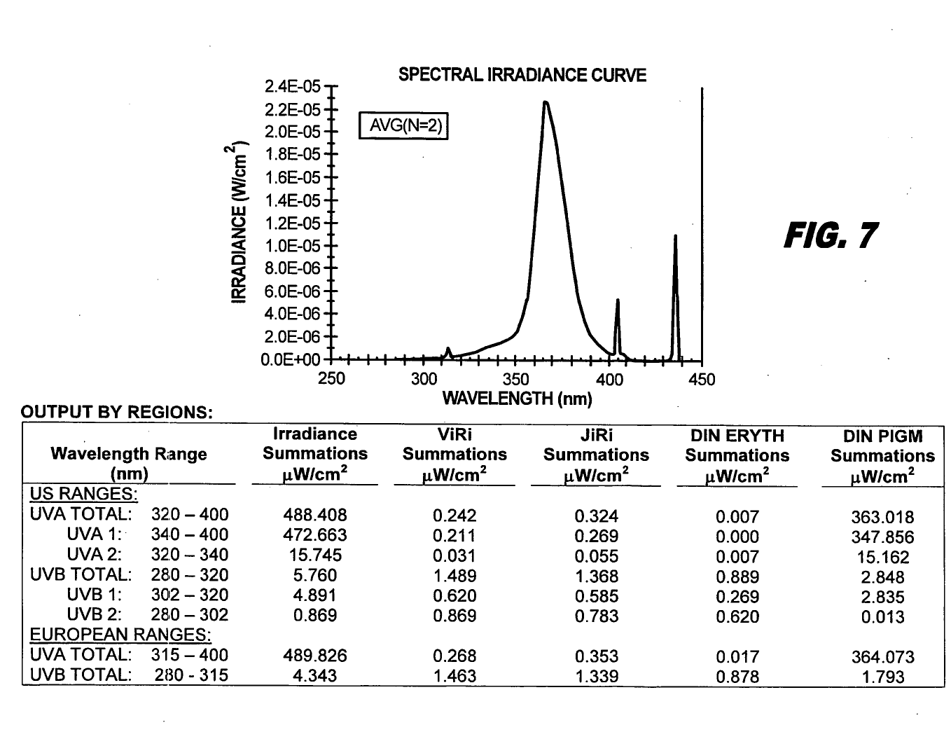

FIG. 3 provides a spectral irradiance curve and irradiance data for a prior art tanning lamp construction and FIGS. 4-7, provide irradiance curves and data summations for exemplary Coatings 1-4, respectfully. Each lamp was constructed with argon gas and mercury contained within the tube and the lamps did not include a reflector coating. The irradiance data which is summarized in the table is provided in units of μW/cm². The measurements were made using an Optronic Laboratories OL 754 double monochrometer spectroradiometer system.

The irradiance data for the lamps of FIGS. 3 through 7 is summarized below in Table 3.

TABLE 3
Summary of Irradiance Date for Exemplary Coatings

| PROPERTY | nm | Prior Art | Coating 1 | Coating 2 | Coating 3 | Coating 4 |
|---|---|---|---|---|---|---|
| UVB | 280-320 | 24.534 | 10.631 | 5.73 | 9.36 | 5.76 |
| UVB1 | 302-320 | 23.616 | 8.775 | 4.868 | 7.789 | 4.891 |
| UVB2 | 280-302 | 0.917 | 1.856 | 0.862 | 1.568 | 0.869 |
| UVA | 320-400 | 933.749 | 819.525 | 489.731 | 491.53 | 488.408 |
| UVA1 | 340-400 | 739.726 | 809.999 | 473.979 | 475.172 | 472.663 |
| UVA2 | 320-340 | 194.023 | 9.526 | 15.752 | 16.359 | 15.745 |
| UVTOTAL | 280-400 | 958.283 | 830.156 | 495.461 | 500.89 | 494.168 |
| % UVB | | 2.56 | 1.28 | 1.16 | 1.87 | 1.17 |
| % UVA | | 97.44 | 98.72 | 98.84 | 98.13 | 98.83 |
| % UVA1 | | 77.19 | 97.57 | 95.66 | 94.87 | 95.65 |
| % UVA2 | | 20.25 | 1.15 | 3.18 | 3.27 | 3.19 |
| UVB2/UVB | | 3.74 | 17.46 | 15.04 | 16.75 | 15.09 |

As illustrated in Table 3, for the prior art lamp tested, the ultraviolet radiation emitted in a range of about 320 nm to about 340 nm was 20.25 percent of the ultraviolet radiation emitted from the lamp in a range of about 280 nm to about 400 nm. Alternatively, for each of the lamps constructed in accordance with certain exemplary embodiments of the present invention, the ultraviolet radiation emitted from these lamps in a range of about 320 nm to about 340 nm is less than 10 percent of the ultraviolet radiation emitted from the lamp in a range of about 280 nm to about 400 nm. In fact, for lamp having coating 1 applied to its inner surface, the UVA2 percentage was as low as 1.15. Such a lamp construction will dramatically reduce the harmful effects caused to the skin by traditional UV exposure.

Additionally, in each of the lamps tested, the ultraviolet radiation emitted from the lamp in UVA1 range is greater than about 85 percent of the total ultraviolet radiation emitted from the lamp. However, in the prior art lamp, the UV radiation emitted from the UVA1 region amounts to 77.19 percent of the total UV emissions.

Table 3 in conjunction with the figures also shows that for the lamps constructed with coatings 1, 2, and 4, the ultraviolet radiation emitted from the lamp in a range of about 280 nm to about 320 nm (the UVB spectrum) is less than about 1.5 percent of the total ultraviolet radiation emitted from the lamp.

It should also be noted that Table 3 illustrates the shifting of energy from the UVB1 region to the UVB2 region in order to improve the efficiency of the lamps constructed in accordance with the teachings of this disclosure. For example, the prior art lamp tested emitted only 3.74% of its UVB energy from the UVB2 region, whereas the lamps constructed in accordance with preferred embodiments of the present invention emit more than 15% of the UVB energy from the UVB2 region, resulting in a more efficient lamp construction.

Although the subject invention has been described with respect to preferred embodiments, those skilled in the art will readily appreciate that changes and modifications may be made thereto without departing from the spirit and scope of the subject invention as defined by the appended claims.

What is claimed is:

1. A discharge lamp for use in tanning applications comprising:
a) an elongated vitreous tube having an outer periphery and axially opposed first and second ends which define an axial length for the tube therebetween;
b) a first electrode assembly associated with the first end of the tube;
c) a second electrode assembly associated with the second end of the tube; and
d) a coating on an interior of the tube for emitting ultraviolet radiation in tanning wavelengths when a voltage is applied across the first and second electrodes;
wherein the ultraviolet radiation emitted from the lamp in a range of about 320 nm to about 340 nm is less than or equal to about 10 percent of the ultraviolet radiation emitted from the lamp in a range of about 280 nm to about 400 nm.

2. A discharge lamp as recited in claim 1, wherein the ultraviolet radiation emitted from the lamp in a range of about 320 nm to about 340 nm is less than about 1 percent of the ultraviolet radiation emitted from the lamp in a range of about 280 nm to about 400 nm.

3. A discharge lamp as recited in claim 1, wherein the ultraviolet radiation emitted from the lamp in a range of about 340 nm to about 400 nm is greater than about 85 percent of the ultraviolet radiation emitted from the lamp in a range of about 280 nm to about 400 nm.

4. A discharge lamp as recited in claim 1, wherein the ultraviolet radiation emitted from the lamp in a range of about 280 nm to about 320 nm is less than about 10 percent of the ultraviolet radiation emitted from the lamp in a range of about 280 nm to about 400 nm.

5. A discharge lamp as recited in claim 4, wherein the ultraviolet radiation emitted from the lamp in a range of about 280 nm to about 320 nm is less than about 1.5 percent of the ultraviolet radiation emitted from the lamp in a range of about 280 nm to about 400 nm.

6. A discharge lamp as recited in claim 1, wherein the ultraviolet radiation emitted from the lamp in a range of about 280 nm to about 302 nm is greater than about 5 percent of the ultraviolet radiation emitted from the lamp in a range of about 280 nm to about 320 nm.

7. A discharge lamp as recited in claim 6, wherein the ultraviolet radiation emitted from the lamp in a range of about 280 nm to about 302 nm is greater than about 10 percent of the ultraviolet radiation emitted from the lamp in a range of about 280 nm to about 320 nm.

8. A discharge lamp as recited in claim 1, wherein the ultraviolet radiation emitted from a lamp in a range of about 320 nm to about 400 nm is greater than about 98 percent of the ultraviolet radiation emitted from the lamp in a range of about 280 nm to about 400 nm.

9. A discharge lamp as recited in claim 8, wherein the ultraviolet radiation emitted from the lamp in a range of about 280 nm to about 320 nm is less than about 1.5 percent of the ultraviolet radiation emitted from the lamp in a range of about 280 nm to about 400 nm.

10. A discharge lamp as recited in claim 1, wherein the coating is applied to the interior of the tube in more than one layer.

11. A discharge lamp as recited in claim 1, wherein the coating is applied to the interior of the tube along at least a portion of the length of the tube.

12. A device for effectuating tanning of a person's skin, the device comprising:
a) a housing structure having a body portion and a base portion, the body portion defining an internal tanning chamber which is adapted and configured for receiving the person;
b) and a discharge lamp assembly disposed within the internal tanning chamber, the lamp assembly including:
i) an elongated vitreous tube having an outer periphery and axially opposed first and second ends which define an axial length for the tube therebetween;
ii) a first electrode assembly associated with the first end of the tube;
iii) a second electrode assembly associated with the second end of the tube; and
iv) a coating on an interior of the tube for emitting ultraviolet radiation in tanning wavelengths when a voltage is applied across the first and second electrodes, wherein the ultraviolet radiation emitted from the lamp in a range of about 320 nm to about 340 nm is less than or equal to about 10 percent of the ultraviolet radiation emitted from the lamp in a range of about 280 nm to about 400 nm.

13. A method of exposing a person to ultraviolet radiation comprising the steps of:
a) positioning a discharge lamp assembly in proximity to a person, the discharge lamp including:
i) an elongated vitreous tube having an outer periphery and axially opposed first and second ends which define an axial length for the tube therebetween;
ii) a first electrode assembly associated with the first end of the tube;
iii) a second electrode assembly associated with the second end of the tube; and
iv) a coating on an interior of the tube for emitting ultraviolet radiation in tanning wavelengths when a voltage is applied across the first and second electrodes, wherein the ultraviolet radiation emitted from the lamp in a range of about 320 nm to about 340 nm is less than or equal to about 10 percent of the ultraviolet radiation emitted from the lamp in a range of about 280 nm to about 400 nm.
b) energizing said discharge lamp for a period of time to effect skin tanning of said person.

14. A phosphor composition for use in a discharge lamp comprising:
a) europium activated strontium tetraborate; and
b) thallium activated calcium zinc orthophosphate.

15. A phosphor composition as recited in claim 14, wherein the composition includes at least about 86% by weight of europium activated strontium tetraborate.

16. A phosphor composition as recited in claim 14, further comprising lead activated barium silicate.

17. A phosphor composition as recited in claim 16, wherein the composition includes about 59% by weight europium activated strontium tetraborate an about 13% by weight thallium activated calcium zinc orthophosphate.

18. A phosphor composition as recited in claim 16, wherein the composition includes about 62% by weight europium activated strontium tetraborate and about 4% by weight thallium activated calcium zinc orthophosphate.

19. A phosphor composition as recited in claim 16, wherein the composition includes about 65% by weight europium activated strontium tetraborate and about 6% by weight thallium activated calcium zinc orthophosphate.

20. A discharge lamp for use in tanning applications comprising:
a) an elongated vitreous tube having an outer periphery and axially opposed first and second ends which define an axial length for the tube therebetween;
b) a first electrode assembly associated with the first end of the tube;
c) a second electrode assembly associated with the second end of the tube; and
d) a coating on an interior of the tube for emitting ultraviolet radiation in taming wavelengths when a voltage is applied across the first and second electrodes;
wherein the ultraviolet radiation emitted from the lamp in a range of about 320 nm to about 340 nm is less than or equal to about 10 percent of the ultraviolet radiation emitted from the lamp in a range of about 280 m to about 400 nm and the ultraviolet radiation emitted from the lamp in the range of about 340 nm to about 400 nm is greater than or equal to about 85% of the total UV emissions for the lamp.

21. A discharge lamp as recited in claim 20, wherein the ultraviolet radiation emitted from the lamp in a range of about 320 nm to about 340 nm is less than about 1 percent of the ultraviolet radiation emitted from the lamp in a range of about 280 nm to about 400 nm.

22. A discharge lamp as recited in claim 20, wherein the ultraviolet radiation emitted from the lamp in a range of about 280 nm to about 320 nm is less than about 10 percent of the ultraviolet radiation emitted from the lamp in a range of about 280 mm to about 400 nm.

23. A discharge lamp as recited in claim 22, wherein the ultraviolet radiation emitted from the lamp in a range of about 280 nm to about 320 nm is less than about 1.5 percent of the ultraviolet radiation emitted from the lamp in a range of about 280 nm to about 400 nm.
